(19) United States
(12) Patent Application Publication
(10) Pub. No.: US 2013/0310945 A1
(43) Pub. Date: Nov. 21, 2013

(54) ACETABULAR SCREW HOLE PLUGS

(71) Applicants: W. Jason Slone, Silver Lake, IN (US); Margaret Kelly, Warsaw, IN (US); Aaron P. Smith, Warsaw, IN (US); Jason D. Meridew, Warsaw, IN (US); Austen Davenport, Columbia City, IN (US); Paige Scott, West Lafayette, IN (US); Randi Holderbaum, Leesburg, VA (US); Jason M. Endsley, Warsaw, IN (US); Mark A. Bollinger, Fort Wayne, IN (US)

(72) Inventors: W. Jason Slone, Silver Lake, IN (US); Margaret Kelly, Warsaw, IN (US); Aaron P. Smith, Warsaw, IN (US); Jason D. Meridew, Warsaw, IN (US); Austen Davenport, Columbia City, IN (US); Paige Scott, West Lafayette, IN (US); Randi Holderbaum, Leesburg, VA (US); Jason M. Endsley, Warsaw, IN (US); Mark A. Bollinger, Fort Wayne, IN (US)

(73) Assignee: BIOMET MANUFACTURING CORP., Warsaw, IN (US)

(21) Appl. No.: 13/473,702

(22) Filed: May 17, 2012

(51) Int. Cl. A61F 2/34

(52) U.S. Cl. 623/22.34

(57) ABSTRACT

An acetabular shell assembly according to the principles of the present disclosure includes a substrate and a screw hole plug. The substrate can have an inner surface, an outer surface, and a screw hole extending through the inner and outer surfaces and configured to receive a screw to fix the substrate to bone within an acetabulum. The screw hole plug can be configured to be selectively retained relative to the substrate.

### FIELD

The present disclosure relates to plugs for screw holes in an acetabular shell.

### BACKGROUND

This section provides background information related to the present disclosure which is not necessarily prior art.

Prosthetic implants used in total hip replacements can include an acetabular shell and a femoral component, with an articular interface therebetween. The acetabular shell is placed in an acetabulum and can define screw holes through which screws can be inserted to fix the acetabular shell to a pelvic bone. Some of the screw holes may be used while others of the screw holes may not be used.

### SUMMARY

This section provides a general summary of the disclosure, and is not a comprehensive disclosure of its full scope or all of its features.

An acetabular shell assembly according to the principles of the present disclosure includes a substrate and a screw hole plug. The substrate can have an inner surface, an outer surface, and a screw hole extending through the inner and outer surfaces and configured to receive a screw to fix the substrate to bone within an acetabulum. The screw hole plug can be configured to be selectively retained relative to the substrate.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of a first acetabular shell and a first screw hole plug according to the principles of the present disclosure;

FIG. 2 is a section view of the first acetabular shell taken along the line 2-2 shown in FIG. 1, and a side view of the first screw hole plug;

FIG. 3 is a partial section view of a second acetabular shell and a side view of a second screw hole plug according to the principles of the present disclosure;

FIG. 4 is a partial section view of a third acetabular shell and a side view of a third screw hole plug according to the principles of the present disclosure;

FIG. 5 is a partial section view of a fourth acetabular shell and a side view of a third screw hole plug according to the principles of the present disclosure;

FIG. 6 is a partial section view of a fifth acetabular shell and a side view of a fifth screw hole plug according to the principles of the present disclosure;

FIG. 7A is a partial section view of a fifth acetabular shell and a side view of a fifth screw hole plug according to the principles of the present disclosure;

FIG. 7B is an end view of the fifth screw hole plug in the direction of the arrow 7B shown in FIG. 7A;

FIG. 7C is a planar view of a screw hole in the fifth acetabular shell in the direction of the arrows 7C shown in FIG. 7A;

FIG. 8A is a partial section view of a sixth acetabular shell and a side view of a sixth screw hole plug according to the principles of the present disclosure;

FIG. 8B is a perspective view of the sixth screw hole plug and a c-clip that fits into a groove in the sixth screw hole plug;

FIG. 8C is a partial section view of the sixth screw hole plug inserted into a screw hole in the sixth acetabular shell, and the c-clip inserted into the groove in the sixth screw hole plug and into a groove in the sixth acetabular shell;

FIG. 9A is a partial section view of a seventh acetabular shell and a side view of a seventh screw hole plug according to the principles of the present disclosure;

FIG. 9B is an end view of the seventh screw hole plug in the direction of the arrow 9B shown in FIG. 9A;

FIG. 10A is a partial section view of an eighth acetabular shell and a side view of a eighth screw hole plug according to the principles of the present disclosure; and

FIG. 10B is an end view of the eighth screw hole plug in the direction of the arrow 10B shown in FIG. 10A.

Corresponding reference numerals indicate corresponding parts throughout the several views of the drawings.

### DESCRIPTION OF VARIOUS EMBODIMENTS

Example embodiments will now be described more fully with reference to the accompanying drawings.

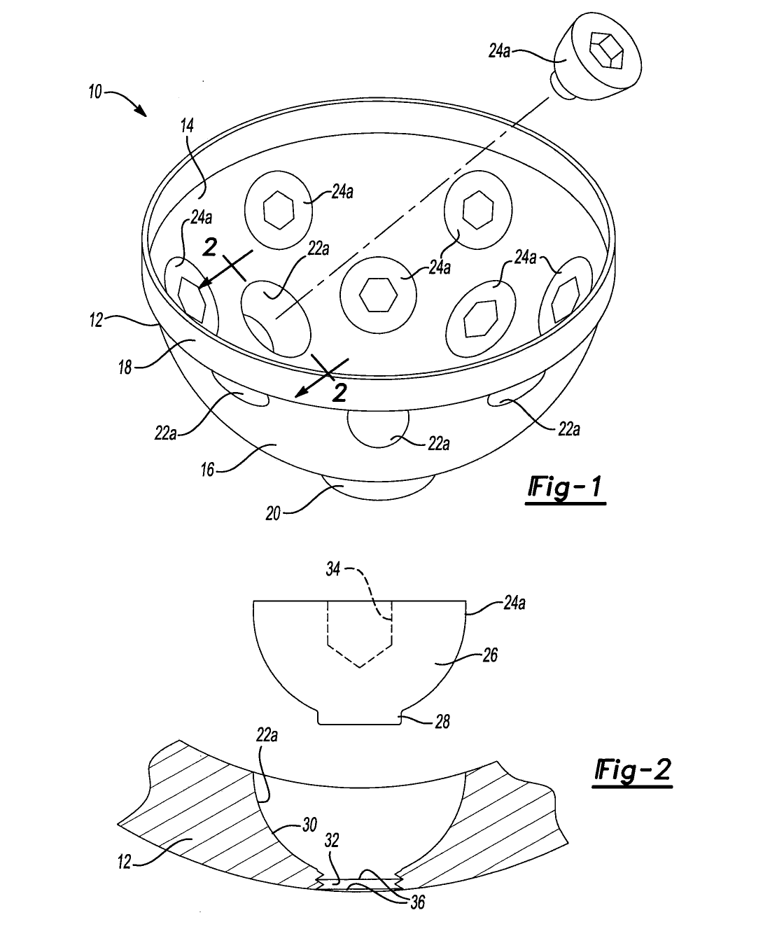

Referring to FIG. 1, an acetabular shell assembly 10 can include an acetabular shell or substrate 12. The substrate 12 and/or a liner placed inside the substrate 12 can be formed from polymer, such as polyurethane, polyethylene, and polyether ether ketone (PEEK). The substrate 12 can be formed from metal or metal powder, such as titanium, cobalt, chrome, and stainless steel. The substrate 12 can be formed from polymer using molding such as injection molding or compression molding. The substrate 12 can be formed from metal or metal powder using machining, forging, casting, and/or isostatic pressing.

The substrate 12 can have a hemispherical shape with an inner surface 14 and an outer surface 16, and the substrate 12 can include a shoulder 18 and a shoulder 20. The shoulder 18 is positioned around the perimeter of the substrate 12 and extends radially from the outer surface 16 of the substrate 12. The shoulder 20 is positioned near the center of the substrate 12 and extends axially from the outer surface 16 of the substrate 12.

The substrate 12 can define one or more screw holes 22a, which can be disposed at various locations on the substrate 12. The screw holes 22a can extend through the inner and outer surfaces 14, 16 of the substrate 12. The screw holes 22a are configured to receive screws to fix the substrate 12 to bone. The screw holes 22a can be threaded, counterbored, and/or countersunk.

The acetabular shell assembly 10 can also include one or more screw hole plugs 24a that are formed separate from the substrate 12 and inserted into the screw holes 22a of the substrate 12 to plug the screw holes 22a. The substrate 12 and the screw hole plugs 24a can be provided separately, and a user, such as a surgeon, can insert the screw hole plugs 24a into those of the screw holes 22a that the user does not use. The user can insert the screw hole plugs 24a before and/or after the substrate is fixed within an acetabulum. Alternatively, the acetabular shell assembly 10 can be provided with the screw hole plugs 24a inserted into the screw holes 22a, and the user can remove the screw hole plugs 24a from those of the screw holes 22a that the user uses or intends to use by passage of a fastener.

The screw hole plugs 24a can be formed from polymer, such as polyurethane, polyethylene, and polyether ether ketone (PEEK). The screw hole plugs 24a can be formed from metal or metal powder, such as titanium, cobalt, chrome, and stainless steel. The screw hole plugs 24a can be formed from polymer using molding such as injection molding or compression molding. The screw hole plugs 24a can be formed from metal or metal powder using machining, forging, casting, selective laser sintering, and/or isostatic pressing.

With additional reference to FIG. 2, the screw hole plugs 24a are configured to plug the screw holes 22a in the substrate 12. The screw hole plugs 24a can include a hemispherical portion 26 and a cylindrical portion 28 sized and shaped to plug a hemispherical portion 30 and a cylindrical portion 32, respectively, of the screw holes 22a. The hemispherical portion 26 of the screw hole plugs 24a can define a socket 34, such as an internal hex, and the cylindrical portion 32 of the substrate 12 can include threads 36.

The material from which the screw hole plugs 24a are formed can be softer and more deformable than the material from which the internal threads 36 are formed. For example, the screw hole plugs 24a can be formed from PEEK and the substrate 12 can be formed from stainless steel. This enables the threads 36 to tap threads into the screw hole plugs 24a as the screw hole plugs 24a are inserted into the screw holes 22a. The socket 34 can be used to rotationally drive the screw hole plugs 24a in the screw holes 22a and thereby thread the screw hole plugs 24a into the screw holes 22a. The screw hole plugs 24a can be threaded into the screw holes 22a until the hemispherical portion 26 of the screw hole plugs 24a seats against the hemispherical portion 30 of the screw holes 22a. This closes and/or seals the screw holes 22a to prevent material, such as liquids or debris, from migrating through the screw holes 22a.

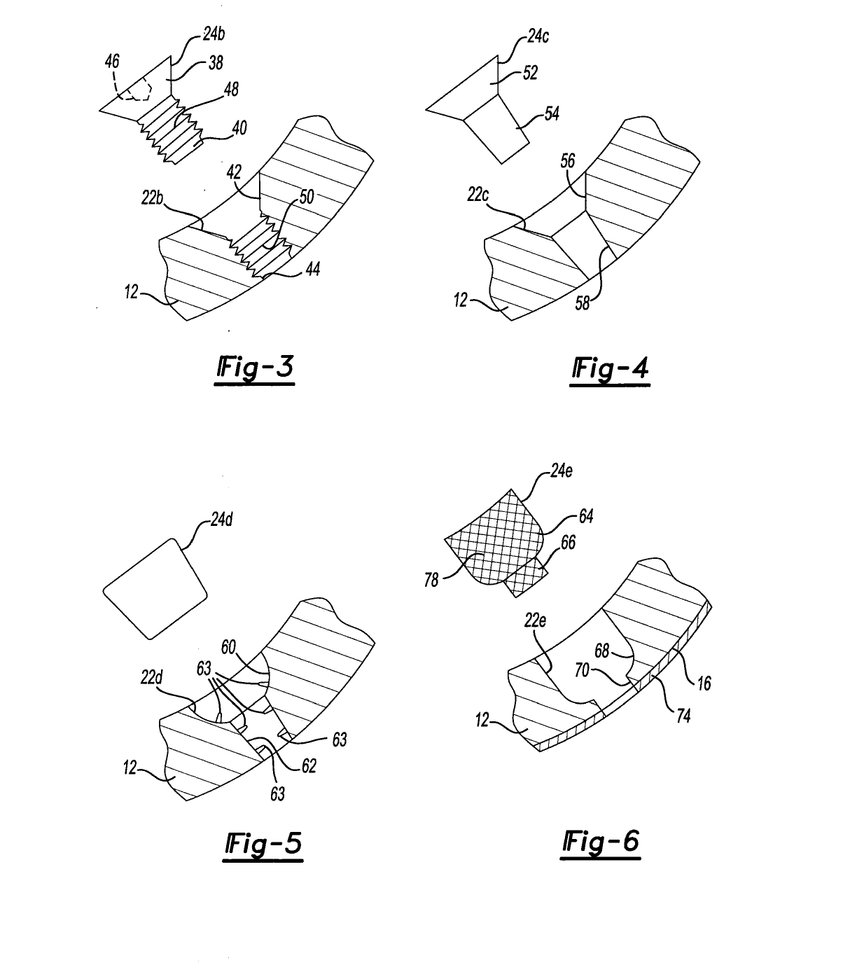

Referring to FIG. 3, the substrate 12 can define one or more screw holes 22b that accommodate one or more screw hole plugs 24b. The screw hole plugs 24b can be formed from the same or different materials as the screw hole plugs 24a and can be provided together with or separate from the substrate 12. However, the screw holes 22b and the screw hole plugs 24b can be configured differently than the screw holes 22a and the screw hole plugs 24a, respectively.

The screw hole plugs 24b are configured to plug the screw holes 22b. The screw hole plugs 24b can include a tapered head 38 and a shank 40 sized and shaped to plug a tapered portion 42 and a cylindrical portion 44, respectively, of the screw holes 22b. The tapered head 38 of the screw hole plugs 24b can define a socket 46, such as an internal hex. The shank 40 of the screw hole plugs 24b can include threads 48 that are configured to mesh or mate with threads 50 defined in the cylindrical portion 44 of the screw holes 22b.

The socket 46 can be used to rotate and drive the screw hole plugs 24b in the screw holes 22b and thereby engage the threads 48 on the screw hole plugs 24b with the threads 50 in the screw holes 22b. The screw hole plugs 24b can be threaded into the screw holes 22b until the tapered head 38 of the screw hole plugs 24b seats against the tapered portion 42 of the screw holes 22b. Also, the taper angles of the tapered head 38 of the screw hole plug 24b and the tapered portion 42 of the screw hole 22b can match and/or can be within a predetermined range (e.g., 3 degrees to 5 degrees) to yield a taper lock. This closes and/or seals the screw holes 22b to prevent material from migrating through the screw holes 22b. An adhesive can be applied to the threads 48 on the screw hole plugs 24b and/or to the threads 50 in the screw holes 22b in order to prevent the screw hole plugs 24b from backing out of the screw holes 22b.

Referring to FIG. 4, the substrate 12 can define one or more screw holes 22c that accommodate one or more screw hole plugs 24c. The screw hole plugs 24c can be formed from the same or different materials as the screw hole plugs 24a and can be provided together with or separate from the substrate 12. However, the screw holes 22c and the screw hole plugs 24c can be configured differently than the screw holes 22a and the screw hole plugs 24a, respectively.

The screw hole plugs 24c are configured to plug the screw holes 22c. The screw hole plugs 24c can include a tapered head 52 and a shank 54 sized and shaped to plug a tapered portion 56 and a cylindrical portion 58, respectively, of the screw holes 22c. The screw hole plugs 24c can be oversized relative to the screw holes 22c. For example, The tapered head 52 and/or the shank 54 of the screw hole plugs 24c can have an outer diameter that is greater than an inner diameter of the tapered portion 56 and/or the cylindrical portion 58, respectively, of the screw holes 22c.

The screw hole plugs 24c can be inserted into the screw holes 22c until the tapered head 52 of the screw hole plugs 24c seats against the tapered portion 56 of the screw holes 22c. When the screw hole plugs 24c are oversized and are inserted into the screw holes 22c, interference between the outer diameter of the screw hole plugs 24c and the inner diameter of the screw holes 22c can yield a press fit. Also, the taper angles of the tapered head 52 of the screw hole plug 24c and the tapered portion 56 of the screw hole 22c can match and/or can be within a predetermined range (e.g., 3 degrees to 5 degrees) to yield a taper lock. The press fit and/or the taper lock can retain the screw hole plugs 24c in the screw holes 22c and close and/or seal the screw holes 22c to prevent material from migrating through the screw holes 22c.

Referring to FIG. 5, the substrate 12 can define one or more screw holes 22d that accommodate one or more screw hole plugs 24d. The screw hole plugs 24d can be provided separate from the substrate 12, and the screw hole plugs 24d can be inserted into those of the screw holes 22d that are not used. The screw holes 22d and the screw hole plugs 24d can be configured differently than the screw holes 22a and the screw hole plugs 24a, respectively.

The screw hole plugs 24d are configured to plug the screw holes 22d. The screw hole plugs 24d can be formed from a malleable, natural or synthetic biocompatible material that can be manipulated to assume various shapes. For example, the screw hole plugs 24d can be formed from bone cement, bone wax, silicone, or biocompatible rubber. In various embodiments, the screw hole plugs 24d can be made from polyethylene, and the screw hole plugs 24d may be heated to soften the screw hole plugs 24d. When the screw hole plugs 24d is pressed into the screw holes 22d, the screw hole plugs 24d assume the shape of the screw holes 22d and retain that shape. This closes and/or seals the screw holes 22d to prevent material from migrating through the screw holes 22d.

Although the screw hole plugs 24d are depicted as having a block shape and the screw holes 22d are depicted as having a rounded portion 60 and a cylindrical portion 62, other shapes are viable as the screw hole plugs 24d are malleable. Also, the screw hole plugs 24d can be larger than the screw holes 22d. Additionally, the substrate 12 can include one or more interference ridges 63 disposed in the screw holes 22d to engage or bite into the screw hole plugs 24d. Furthermore, the screw hole plugs 24d can assume and retain the shape of the screw holes 22d when the screw hole plugs 24d are pressed into the screw holes 22d in an axial direction only.

Referring to FIG. 6, the substrate 12 can define one or more screw holes 22e that accommodate one or more screw hole plugs 24e. The screw hole plugs 24e can be formed from the same or different materials as the screw hole plugs 24a and can be provided together with or separate from the substrate 12. However, the screw holes 22e and the screw hole plugs 24e can be configured differently than the screw holes 22a and the screw hole plugs 24a, respectively.

The screw hole plugs 24e are configured to plug the screw holes 22e. The screw hole plugs 24e can include a rounded portion 64 and a boss 66 sized and shaped to plug a rounded portion 68 and a cylindrical portion 70, respectively, of the screw holes 22e. An outer surface 72 of the screw hole plugs 24e can be ridged, unsmooth, and/or knurled, and an outer diameter of the screw hole plugs 24e can be greater than an inner diameter of the screw holes 22e.

The screw hole plugs 24e can be inserted into the screw holes 22e until the rounded portion 64 of the screw hole plugs 24e seats against the rounded portion 68 of the screw holes 22e. When the screw hole plugs 24e are inserted into the screw holes 22e, the ridged, unsmooth, and/or knurled outer surface 72 of the screw hole plugs 24e can deform or project into the substrate 12 to yield an interference fit between the screw hole plugs 24e and the substrate 12. This interference fit can retain the screw hole plugs 24e in the screw holes 22e and close and/or seal the screw holes 22e to prevent material from migrating through the screw holes 22e.

The substrate 12 and the screw hole plugs 24e can be formed from different materials so that one is harder than the other. The screw hole plugs 24e can be formed from a harder material than the substrate 12 so that the knurled outer surface 72 of the screw hole plugs 24e deforms the substrate 12. Alternatively, the substrate 12 can be formed from a harder material than the screw hole plugs 24e and can include a projection that deforms the screw hole plugs 24e as the screw hole plugs 24e are inserted into the screw holes 22e. In other embodiments, the substrate 12 and the screw hole plugs 24e can be formed from materials that have the same hardness and can deform each other as the screw hole plugs 24e are inserted into the screw holes 22e. The deformation of both the substrate 12 and the screw hole plugs 24e may yield a cold weld that retains the screw hole plugs 24e within the screw holes 22e.

In addition, a coating 74 can be applied to the outer surface 16 of the substrate 12 before or after the screw hole plugs 24e are inserted. The coating 74 can be a coating that promotes bone ingrowth or bone ongrowth, such as a porous coating, a plasma spray, or a titanium foam. The coating 74 can cover the screw holes 22e, in which case the portion of the coating 74 covering the screw holes 22e can be ruptured and, if necessary, the screw hole plugs 24e can be removed to insert screws therein. Although not shown, the coating 74 can be applied to the substrate 12 in the implementations of FIGS. 1 through 5.

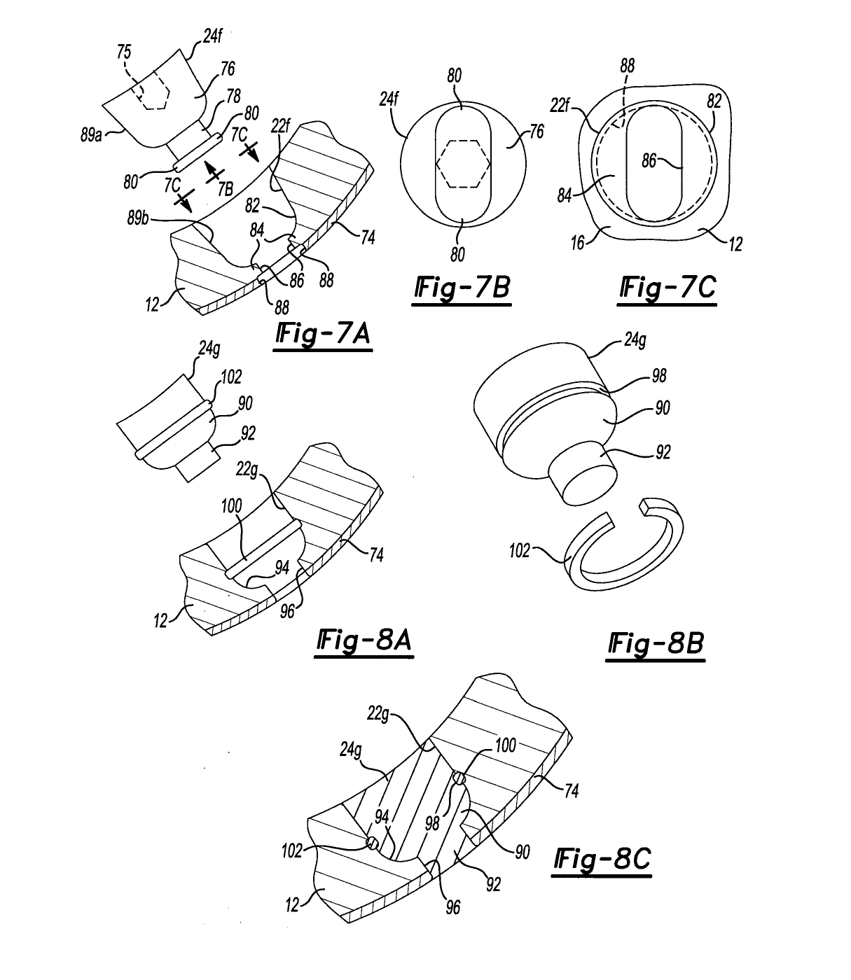

Referring to FIG. 7A through 7C, the substrate 12 can define one or more screw holes 22f that accommodate one or more screw hole plugs 24f. The screw hole plugs 24f can be formed from the same or different materials as the screw hole plugs 24a and can be provided together with or separate from the substrate 12. However, the screw holes 22f and the screw hole plugs 24f can be configured differently than the screw holes 22a and the screw hole plugs 24a, respectively.

The screw hole plugs 24f are configured to plug the screw holes 22f. The screw hole plugs 24f can include a socket 75, such as an internal hex or noncircular shape, a rounded portion 76, a neck 78, and ears 80. The screw holes 22f can include a rounded portion 82, and a locking feature 84 disposed in the screw holes 22f can define a slot 86, and a recess 88. The slot 86 can be shaped so that the ears 80 can be inserted through the slot 86 with a selected clearance, including minimal or no clearance between the ears 80 and the slot 86.

Before inserting the screw hole plugs 24f into the screw holes 22f, the screw hole plugs 24f may be rotated 90 degrees about a longitudinal axis of the screw hole plugs 24f relative to the orientation shown in FIG. 7A. This orientation aligns the ears 80 and the slot 86 so that the ears 80 can be inserted through the slot 86 and into the recess 88. After the ears 80 are inserted, the screw hole plugs 24f can be rotated, for example, using the socket 75, to rotate the ears 80 within the recess 88. Once the ears 80 are rotated within the recess 88, the locking feature 84 retains the ears 80 within the recess 88 and thereby retains the screw hole plugs 24f in the screw holes 22f. The retention of the ears 80 in the recess 88 can lock the screw hole plug 24f in the screw hole 22f.

The underside surface of the locking feature 84 defining the recess 88 can be wedged to move the ears 80 to within the recess 88 and/or to yield a press fit as the screw hole plugs 24f are rotated to lock or retain the ears 80 within the recess 88. Additionally, the screw hole plugs 24f can include a tapered portion 89a and the screw holes 22f can include a tapered portion 89b. The taper angles of the tapered portions 89a, 89b can match and/or can be within a predetermined range (e.g., 2 degrees to 3 degrees) to yield a taper lock. If the underside surface of the locking feature 84 is wedged, the taper lock may be strengthened as the screw hole plug 24f is rotated and the wedged surface of the locking feature 84 draws the screw hole plug 24f into the screw hole 22f.

Referring to FIGS. 8A through 8C, the substrate 12 can define one or more screw holes 22g that accommodate one or more screw hole plugs 24g. The screw hole plugs 24g can be formed from the same or different materials as the screw hole plugs 24a and can be provided together with or separate from the substrate 12. However, the screw holes 22g and the screw hole plugs 24g can be configured differently than the screw holes 22a and the screw hole plugs 24a, respectively.

The screw hole plugs 24g are configured to plug the screw holes 22g. The screw hole plugs 24g can include a rounded portion 90 and a cylindrical portion 92 shaped and sized to plug a rounded portion 94 and a cylindrical portion 96, respectively, of the screw holes 22g. The screw hole plugs 24g can define a groove 98 that extends around the perimeter of the screw hole plugs 24g, and the screw holes 22g can include a groove 100 that extends around the perimeter of the screw holes 22g. A locking ring 102, such as a c-clip or a ball seal spring, can be inserted into the groove 98 in the screw hole plugs 24g or into the groove 100 in the screw holes 22g before the screw hole plugs 24g are inserted into the screw holes 22g. The locking ring 102 can be integral to the screw hole plugs 24g. Alternatively, the locking ring 102 can be separate from the screw hole plugs 24g and the locking ring 102 can be fixed to the screw hole plugs 24g. The locking ring 102 can be formed from an elastic material.

The screw hole plugs 24g can be inserted into the screw holes 22g until the rounded portion 90 of the screw hole plugs 24g seats against the rounded portion 94 of the screw holes 22g (FIG. 8C). At that point, the groove 98 in the screw hole plugs 24g can align with the groove 100 in the screw hole 22g. This allows the locking ring 102 to relax or snap into the grooves 98, 100, and thereby retain or lock the screw hole plugs 24g in the screw holes 22g. The locking ring 102 can deform when the screw hole plugs 24g are inserted into the screw holes 22g and contract into the groove 98 or expand into the groove 100 when the grooves 98, 100 are aligned and the screw hole plugs 24g are seated.

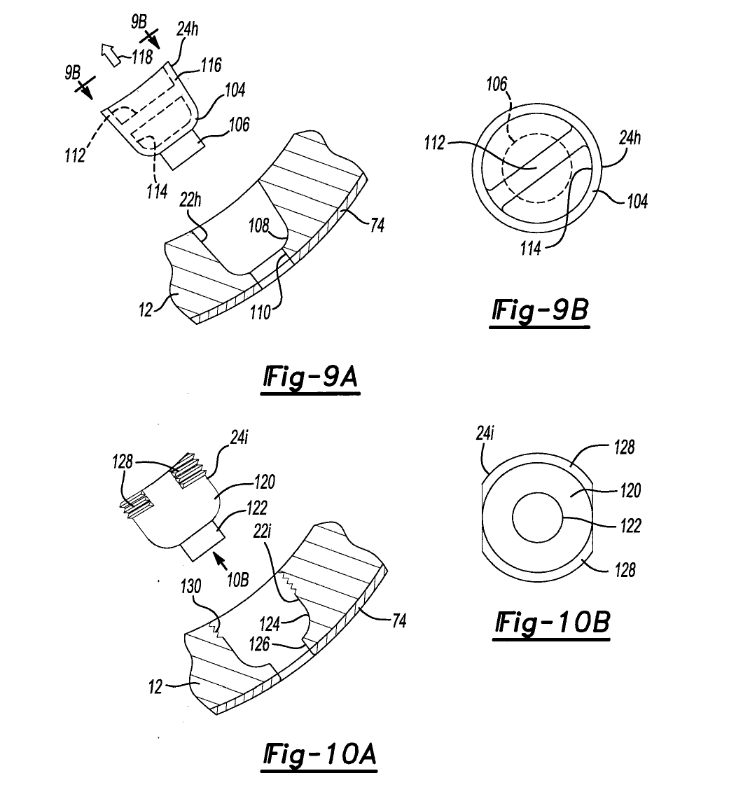

Referring to FIGS. 9A and 9B, the substrate 12 can define one or more screw holes 22h that accommodate one or more screw hole plugs 24h. The screw hole plugs 24h can be formed from the same or different materials as the screw hole plugs 24a and can be provided together with or separate from the substrate 12. However, the screw holes 22h and the screw hole plugs 24h can be configured differently than the screw holes 22a and the screw hole plugs 24a, respectively.

The screw hole plugs 24h are configured to plug the screw holes 22h. The screw hole plugs 24h can include a rounded portion 104 and a cylindrical portion 106 shaped and sized to plug a rounded portion 108 and a cylindrical portion 110, respectively, of the screw holes 22h. The screw hole plugs 24h can be hollow, and a bar 112 can extend across a hollow portion 114 of the screw hole plugs 24h defined by a thin wall 116. The outer diameter of the thin wall 116 can be slightly greater than the inner diameter of the screw holes 22h, and the thin wall 116 can collapse to press or seal into the screw holes 22h.

The screw hole plugs 24h can be inserted into the screw holes 22h until the rounded portion 104 of the screw hole plugs 24g seats against the rounded portion 108 of the screw holes 22h. Once the screw hole plugs 24h are inserted into the screw holes 22h, the press fit between the screw hole plugs 24h and the substrate 12 retains the screw hole plugs 24h in the screw holes 22h. The screw hole plugs 24h can be removed by inserting a tool, such as pliers, a lever, or a rod with a hook, into the hollow portion 114 of the screw hole plugs 24h and pulling the bar 112 in the general direction of arrow 118.

Referring to FIGS. 10A and 10B, the substrate 12 can define one or more screw holes 22i that accommodate one or more screw hole plugs 24i. The screw hole plugs 24i can be formed from the same or different materials as the screw hole plugs 24a and can be provided together with or separate from the substrate 12. However, the screw holes 22i and the screw hole plugs 24i can be configured differently than the screw holes 22a and the screw hole plugs 24a, respectively.

The screw hole plugs 24i are configured to plug the screw holes 22i. The screw hole plugs 24i can include a rounded portion 120 and a cylindrical portion 122 shaped and sized to plug a rounded portion 124 and a cylindrical portion 126, respectively, of the screw holes 22i. In addition, the screw hole plugs 24i can include threads 128 configured to mate with threads 130 in the screw holes 22i.

The threads 128, 130 can be configured so that the screw hole plugs 24h can be inserted into the screw holes 22h until the rounded portions 104, 108 almost contact each other before the threads 128, 130 start to engage. For example, the threads 128, 130 can extend only partially around a distal portion of the screw hole plugs 24h and the screw holes 22h, respectively. The threads 128, 130 can also be configured so that rotating the screw hole plugs 24i less than one 360 degree turn (e.g., 90 degrees) fully engages the threads 128, 130 and the rounded portions 104, 108. The threads 128, 130 may be fully engaged when the one of the threads 128, 130 contacts a stop included in the other one of the threads 128, 130. This retains the screw hole plugs 24i within the screw holes 22i. The screw hole plugs 24i can include a socket, such as an internal hex or a straight slot, that can be used to rotate the screw hole plugs 24i.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. An acetabular shell assembly, comprising:
a substrate having an inner surface, an outer surface, and a screw hole extending through the inner and outer surfaces and configured to receive a screw to fix the substrate to bone within an acetabulum; and
a screw hole plug configured to at least partially fill the screw hole;
wherein one of the substrate and the screw hole plug is configured to deform a first surface of the other one of the substrate and the screw hole plug by contacting the first surface when the screw hole plug is inserted into the screw hole; and
wherein the screw hole includes a first hemispherical portion and a first straight cylindrical portion, and the screw hole plug includes a second hemispherical portion and a second straight cylindrical portion sized and shaped to plug the first hemispherical portion and the first straight cylindrical portion, respectively.

2. The acetabular shell assembly of claim 1, wherein the screw hole plug is formed from a malleable material that conforms to the shape of the screw hole when the screw hole plug is pressed into the screw hole in an axial direction only.

3. The acetabular shell assembly of claim 2, wherein the malleable material is at least one of bone cement, bone wax, silicone, polyethylene, or biocompatible polymer.

4. The acetabular shell assembly of claim 2, wherein the screw hole plug is larger than the screw hole.

5. The acetabular shell assembly of claim 1, wherein the substrate includes threads disposed in the screw hole and configured to tap into the screw hole plug as the screw hole plug is driven into the screw hole.

6. The acetabular shell assembly of claim 5, wherein the substrate is formed from a first material and the screw hole plug is formed from a second material that is softer than the first material.

7. The acetabular shell assembly of claim 5, wherein the threads are disposed in the first cylindrical portion of the screw hole and are configured to tap the second cylindrical portion of the screw hole plug.

8. The acetabular shell assembly of claim 1, wherein the screw hole plug has a knurled outer surface.

9. The acetabular shell assembly of claim 1, wherein the substrate is formed from a first material and the screw hole plug is formed from a second material that is one of harder or softer than the first material.

10. The acetabular shell assembly of claim 1, wherein the substrate is formed from a first material and the screw hole plug is formed from a second material that is identical in hardness to the first material.

11. (canceled)

12. The acetabular shell assembly of claim 11, wherein the screw hole plug includes a wall defining the hollow portion that deforms when the screw hole plug is inserted into the screw hole while still allowing access to the bar.

13. An acetabular shell assembly, comprising:
a substrate having an inner surface, an outer surface, and a screw hole extending through the inner and outer surfaces and configured to receive a screw to fix the substrate to bone, the substrate including a locking feature disposed in the screw hole and the locking feature defines a slot and a recess; and
a screw hole plug including ears, the locking feature retaining the ears in the recess when the ears are inserted through the slot and the screw hole plug is rotated to rotate the ears within the recess.

14. (canceled)

15. The acetabular shell assembly of claim 13, wherein the screw hole plug includes a first tapered portion having a first taper angle and the screw hole includes a second tapered portion having a second taper angle that matches the first taper angle.

16.-20. (canceled)

21. An acetabular shell assembly, comprising:
a substrate having an inner surface, an outer surface, a screw hole extending through the inner and outer surfaces and configured to receive a screw to fix the substrate to bone, and first threads disposed in the screw hole; and
a screw hole plug including second threads configured to fully engage the first threads and thereby retain the screw hole plug within the screw hole when the screw hole plug is inserted into the screw hole and rotated less than one 360 degree turn.

22. (canceled)

23. The acetabular shell assembly of claim 21, wherein the screw hole includes a first rounded portion and a first cylindrical portion, the screw hole plug includes a second rounded portion and a second cylindrical portion, and the second rounded portion seats against the first rounded portion when the second threads fully engage the first threads.

24. An acetabular shell assembly, comprising:
a substrate having an inner surface spaced a distance from an outer surface, and a screw hole extending through the inner surface and outer surface of the substrate, wherein the screw hole is configured to receive a screw to fix the substrate to a bone within an acetabulum; and
a screw hole plug configured to at least partially fill the screw hole;
wherein the substrate is configured to deform at least a first portion of the screw hole plug by contacting the first portion when the screw hole plug is inserted into the screw hole;
wherein the screw hole includes a hole curved portion near the inner surface and a hole straight cylindrical portion near the outer surface; and
wherein the screw hole plug includes a plug curved portion and a plug cylindrical portion sized and shaped to plug the respective hole curved portion and the hole cylindrical portion.

25.-26. (canceled)

27. The acetabular shell assembly of claim 24, wherein the substrate further defines a thread extending into the hole cylindrical portion such that the thread taps a plug thread into the screw hole plug as the screw hole plug is inserted into the screw hole.

28. The acetabular shell assembly of claim 27, wherein the substrate is formed from at least one of a polymer, a metal, or a metal powder.

29. The acetabular shell assembly of claim 28, wherein the screw hole plug is formed from a polymer.

30. The acetabular shell assembly of claim 28, wherein the screw hole plug is formed from at least one of a metal or a metal powder.

31. The acetabular shell assembly of claim 7, wherein the tapped threads are configured to screw the screw hole plug second cylindrical portion into the threads of the first cylindrical portion of the screw hole until the second hemispherical portion of the screw hole plug seats against the first hemispherical portion of the screw holes to close and/or seal the screw hole.
